(19) United States
(12) Patent Application Publication
(10) Pub. No.: US 2023/0166048 A1
(43) Pub. Date: Jun. 1, 2023

(54) PROTECTIVE CAP FOR A SYRINGE NEEDLE

(71) Applicant: Stevanato Group S.P.A., PIOMBINO DESE (PD) (IT)

(72) Inventors: Stefan Heitmann, Hille (DE); Stefan Penner, Bad Oeynhausen (DE)

(21) Appl. No.: 18/059,168

(22) Filed: Nov. 28, 2022

(30) Foreign Application Priority Data
Nov. 26, 2021 (IT) 202021000005552

(51) Int. Cl. A61M 5/32; A61M 5/31

(52) U.S. Cl. CPC A61M 5/3202; A61M 5/3137

(57) ABSTRACT

A protective cap for a syringe needle includes a main body which extends circumferentially about a main axis and having a predetermined axial extension, wherein the main body comprises first gripping surface elements arranged at a first cylindrical portion of the main body and distributed throughout the first cylindrical portion circumferentially spaced apart from each other.

### CROSS REFERENCE

This application claims priority to Italian Application No. 202021000005552 filed on Nov. 22, 2021, the disclosure of which is expressly incorporated by reference herein in its entirety.

### TECHNICAL FIELD

The present invention relates to a protective cap for a syringe needle.

### BACKGROUND

As known, syringes used in the medical/pharmaceutical field are provided with a protective cap removably fitted on the syringe needle to avoid injuring the users.

Protective caps are known consisting of an outer body and an inner body coupled to each other, wherein the outer body is made of a material having a greater rigidity than the material the inner body is made of. This protective cap is also known as “rigid needle shield” (RNS).

On the outer body, typically, surface elements are present that are adapted to facilitate gripping the protective cap.

The Applicant has felt the need to improve the grip of the RNS-type protective caps and has identified a particular arrangement of the gripping surface elements that is suitable to meet this need.

### SUMMARY

Therefore, the present invention relates to a protective cap for a syringe needle, comprising a main body extending circumferentially about a main axis and having a predetermined axial extension, wherein the main body comprises first gripping surface elements arranged at a first cylindrical portion of the main body and distributed, throughout said first cylindrical portion, circumferentially spaced apart from each other.

In the present description and subsequent claims, the terms “circumferentially”, “circumferential” and the like refer to a circumferential direction about the main axis of the main body, the terms “radially”, “radial” and the like refer to a direction orthogonal to the main axis of the main body, while the terms “axially”, “axial” and the like refer to a direction parallel to, or coincident with, the main axis of the main body.

The Applicant has verified that the distribution of the first gripping surface elements throughout the circumferential extension of the main body enables an effective and comfortable grip of the protective cap and, consequently, an easy and safe handling thereof, whatever the contact position of the user’s fingers on the main body is.

Preferably, said first cylindrical portion is a distal portion of the main body with respect to a patient when the protective cap is coupled to the needle and the tip of the needle is oriented towards the patient.

In the above-described operating configuration, therefore, the aforesaid distal cylindrical portion is in a position farther from the patient, or closer to the syringe user, than a proximal cylindrical portion of the main body.

Preferably, said distal first cylindrical portion has a larger diameter than said proximal cylindrical portion. Thereby, the first gripping surface elements are arranged in a sufficiently large and robust portion of the main body, to the benefit of the convenience of use and of the gripping efficiency.

Preferably, each of said first gripping surface elements is arranged between two respective recesses which extend substantially parallel to the main axis, and thus substantially parallel to the direction of removal of the protective cap from the needle, so that any slipping of the user’s fingers on the first gripping surface elements during the removal of the protective cap would not cause the user any particular discomfort to the touch.

Preferably, said first gripping surface elements are circumferentially equally spaced (or substantially equally spaced) apart from each other. Thereby, the effectiveness of the grip is identical whatever the contact position of the user’s fingers on the main body is.

Preferably, the main body comprises second gripping surface elements arranged at a second cylindrical portion of the main body. Such second gripping surface elements help to ensure an effective grip of the protective cap, allowing an easy removal of the protective cap from the needle and an easy repositioning of the protective cap on the needle even when the user’s fingers only partially contact the first gripping surface elements or do not contact them at all.

Preferably, said second gripping surface elements extend substantially parallel to the main axis, thereby achieving the same advantages to the touch as mentioned above with reference to the first gripping surface elements.

Preferably, said second gripping surface elements are grouped in two or three areas which are circumferentially spaced apart from each other, to form two or three groups of second gripping surface elements respectively. In fact, the Applicant considers that, given the effectiveness of the grip offered by the first gripping surface elements, it is possible to limit the circumferential distribution of the second gripping surface elements on the main body, with consequent simplifications in shape and construction.

More preferably, said areas are circumferentially equally spaced (or substantially equally spaced) apart from each other. Thereby the arrangement of the second gripping surface elements is limited to the zones of the protective cap which the user’s fingers typically contact in those cases, which by the way are the most frequent, wherein the user handles the protective cap with two or three fingers (typically the thumb, the index finger and possibly the middle finger).

Preferably, said second cylindrical portion is a proximal portion of the main body with respect to a patient when the protective cap is coupled to the needle and the needle tip is oriented towards the patient.

Preferably, the main body comprises, at the second cylindrical portion, two or three through openings, each one arranged between two respective second gripping surface elements.

The through openings allow the inlet of a sterilising gas, e.g. ethylene oxide, during the sterilisation process of the syringe.

The Applicant has considered to provide a limited number of through openings (two or three) in order not to excessively weaken the main body and to ensure a sufficient gripping area.

Preferably, each through opening has an axial extension lower than or equal to 25% of the axial extension of the main body. This provision also aims at not excessively weakening the main body, while ensuring the passage of ethylene oxide during the sterilisation of the syringe. According to the Applicant, in fact, a greater number of through openings and/or a greater axial extension of the through openings with respect to what described above would excessively weaken the main body, with the risk of bending the protective cap while it is removed from the needle or is repositioned on the needle.

Furthermore, considering that the conventional protective caps, as well as that of the present invention, are typically made by injection moulding of plastic material, a limited number of through openings and/or a limited axial extension thereof eases the production of the protective cap of the present invention, since the mould cavities are more easily filled by the plastic material injected into the mould.

Preferably, the through openings and the groups of second gripping surface elements have a substantially equal circumferential extension. For example, in the case of three groups of second gripping surface elements and three through openings, each through opening and each group of second gripping surface elements has a circumferential extension substantially equal to about ⅙ of the entire circumference, i.e., each through opening and each group of second gripping surface elements have an angular extension equal to about 60°.

Preferably, the protective cap comprises a secondary body extending circumferentially about said main axis in a radially inner position with respect to said main body and having an axial extension lower than that of said main body.

Preferably, the secondary body is made of a material that is permeable to gases, in particular to the ethylene oxide used in the syringe sterilisation process.

More preferably, the secondary body is made of a thermoplastic polymeric elastomer.

Preferably, the secondary body is coupled to the main body by hot moulding a distal lip of the main body on a distal end portion of the secondary body.

Preferably, the distal lip is formed by folding with an undercut a distal end of the main body after softening it by heating, before or after the assembly of the main body on the secondary body.

Preferably, said secondary body comprises a plurality of surface ribs in contact with a radially inner surface of the main body. Thereby, during the sterilisation of the syringe, the ethylene oxide is distributed in the gap formed between the main body and the secondary body subsequently entering inside the secondary body and sterilising the needle and the end portion of the syringe to which the needle is coupled.

Preferably, the ribs of said plurality of surface ribs are substantially parallel to said main axis. Thereby, the assembly of the main body on the secondary body, which is done by relative sliding in the axial direction, is facilitated.

Preferably, the ribs of said plurality of surface ribs are circumferentially equally spaced (or substantially equally spaced) apart from each other. Thereby, during the sterilisation of the syringe, the ethylene oxide is substantially evenly distributed in the gap formed between the main body and the secondary body.

Preferably, the secondary body comprises a plurality of surface recesses, each one arranged between two respective ribs of said plurality of surface ribs.

Preferably, the surface recesses are provided at a proximal portion of the secondary body.

More preferably, the surface recesses extend up to a proximal free end portion of the secondary body.

The surface recesses allow the weight of the protective cap to be contained, as a consequence of the reduction in its mass, and, at the same time, a more even mass distribution. As a result of such reduction and more even mass distribution, the risk of formation of surface defects of the so-called “sink mark” type following moulding is also reduced or avoided.

In addition, the above-mentioned surface recesses reduce or avoid the risk of have the so-called “free jetting”, as the recesses help to direct the flow within the mould, allowing to form an oriented flow front and to control the mould filling step.

The provision of recesses also allows a better cooling of the mould.

### BRIEF DESCRIPTION OF THE DRAWINGS

Further features and advantages of the present invention will become clearer from the following detailed description of a preferred embodiment thereof, made with reference to the appended drawings and provided for indicative and non-limiting purposes. In such drawings:

FIG. 1 is a schematic perspective view of a protective cap for syringe needle according to the present invention;

FIG. 2 is a further schematic perspective view of the protective cap for syringe needle of FIG. 1, taken from another vantage point;

FIG. 3 is a schematic perspective view of an axial section of a first component (main body) of the protective cap for syringe needle of FIG. 1;

FIG. 4 is a schematic perspective view of a second component (secondary body) of the protective cap for syringe needle of FIG. 1;

FIG. 5 is a schematic axial section of a distal portion of the protective cap for syringe needle of FIG. 1.

### DETAILED DESCRIPTION

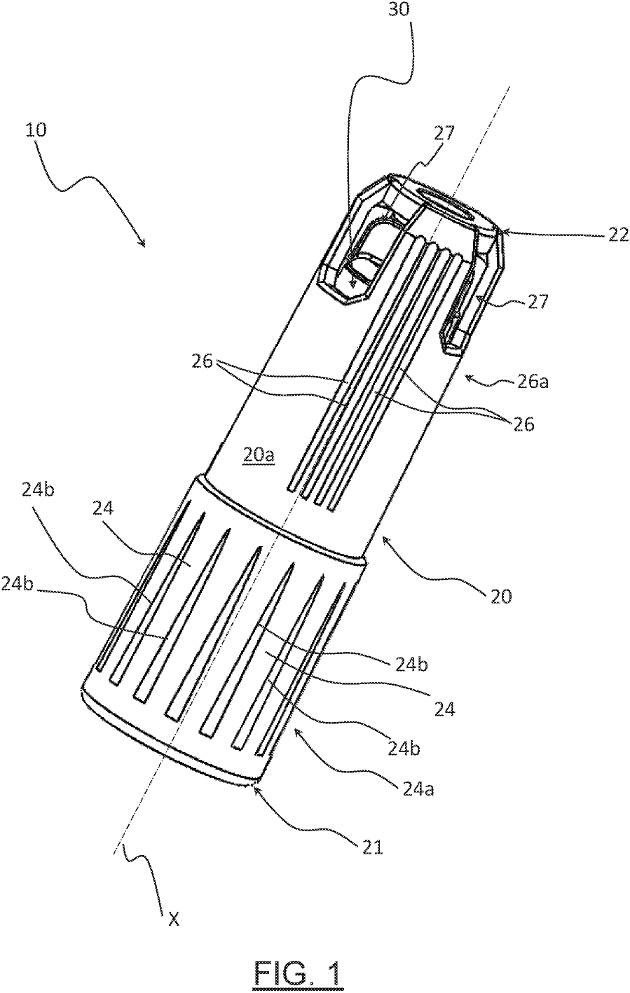

Referring to FIG. 1, a protective cap for a syringe needle according to the present invention is indicated with 10.

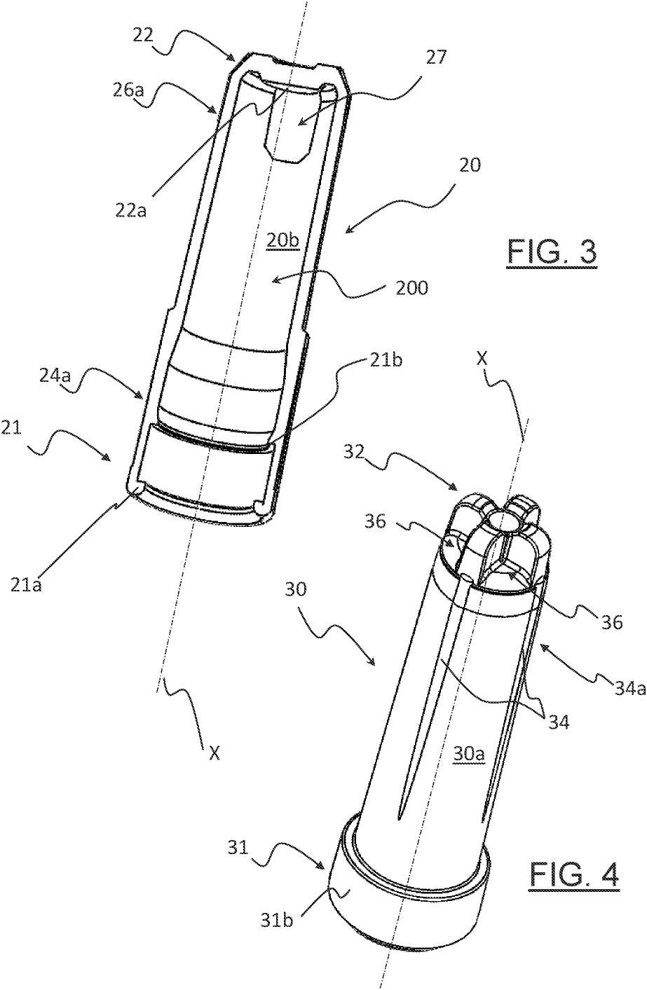

The protective cap 10 is of the “rigid needle shield” (RNS) type and comprises a main body 20 and a secondary body 30, which are shown separately in FIGS. 3 and 4, respectively.

The secondary body 30 is housed in a radially inner position with respect to the main body 20.

The main body 20 and the secondary body 30 are mutually coupled so as to be fixed to each other.

The main body 20 has a substantially cylindrical shape and extends circumferentially about a main axis X.

The main body 20 has a predetermined axial extension.

The main body 20 comprises a cavity 200 open at a distal end portion 21 of the main body 20 and closed at a proximal end portion 22 of the main body 20. Such a cavity 200 is configured to house the secondary body 30.

The main body 20 is preferably made of a rigid thermoplastic material, for example polypropylene.

The secondary body 30 has a substantially cylindrical shape and extends circumferentially about the main axis X.

The secondary body 30 has an axial extension smaller than that of the main body 20.

The secondary body 30 comprises a cavity (not visible in the drawings) open at a distal end portion 31 of the secondary body 30 and closed at a proximal end portion 32 of the secondary body 30. Such a cavity is configured to house the syringe needle.

The secondary body 30 is made of a material permeable to the gases (in particular to the ethylene oxide), for example a thermoplastic polymeric elastomer.

The secondary body 30 has a rigidity lower than that of the material from which the main body 20 is made of.

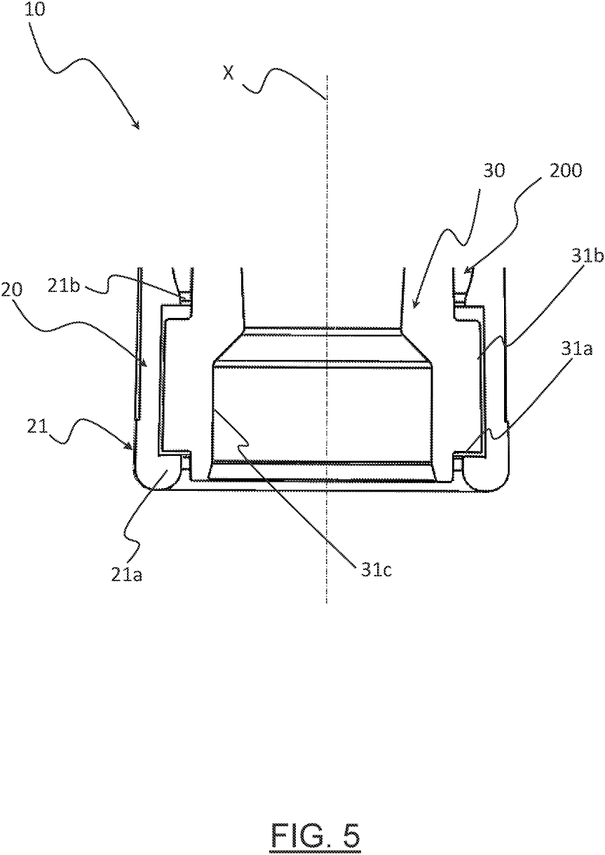

The secondary body 30 is connected to the main body 20 by means of an undercut coupling, as shown in FIG. 5.

In particular, the distal end portion 21 of the main body 20 comprises a distal lip 21a which extends circumferentially and engages with a circumferential seat 31a formed in the distal end portion 31 of the secondary body 30.

A circumferential rib 21b is provided inside the distal end portion 21 of the main body 20, creating a section narrowing and defining an inner diameter smaller than the external diameter of a cylindrical portion 31b with an increased-diameter formed at the distal end portion 31 of the secondary body 30.

As shown in FIGS. 3 and 4, in this configuration the proximal end portion 32 of the secondary body 30 abuts against an abutment surface 22a defined at the proximal end portion 22 of the main body 20, internally to the cavity 200 of the main body 20.

The distal lip 21a is formed by folding the distal end portion 21 of the main body 20 towards the cavity 200 after softening it by heating or hot moulding, before or after the assembly of the main body 20 on the secondary body 30.

The distal end portion 31 of the secondary body 30 comprises an annular portion 31c configured to removably house the cylindrical support (not shown) of the syringe needle.

The radially outer surface 20a of the main body 20 comprises first gripping surface elements 24 and second gripping surface elements 26.

The first gripping surface elements 24 are arranged at a first cylindrical portion 24a of the main body 20. The first cylindrical portion 24a is a distal portion of the main body 20 and is adjacent to the aforementioned distal end portion 21.

The second gripping surface elements 26 are arranged at a second cylindrical portion 26a of the main body 20. The second cylindrical portion 26a is a proximal portion of the main body 20 and is adjacent to the aforementioned proximal end portion 22.

The first cylindrical portion 24a has a diameter larger than that of the second cylindrical portion 26a.

The first gripping surface elements 24 are distributed throughout the first cylindrical portion 24a circumferentially equally spaced apart from each other.

Each of the first gripping surface elements 24 is defined between two respective recesses 24b having an elongated shape and which extend substantially parallel to the main axis X. Such recesses 24b are tapered towards the proximal end portion 22 of the main body 20. Thus, the first gripping surface elements 24 correspond to parts of the first cylindrical portion 24a of the main body 20 which protrude with respect to the recesses 24b upon making the recesses 24b.

The second gripping surface elements 26 are also defined by ribs having an elongated shape and which extend substantially parallel to the main axis X.

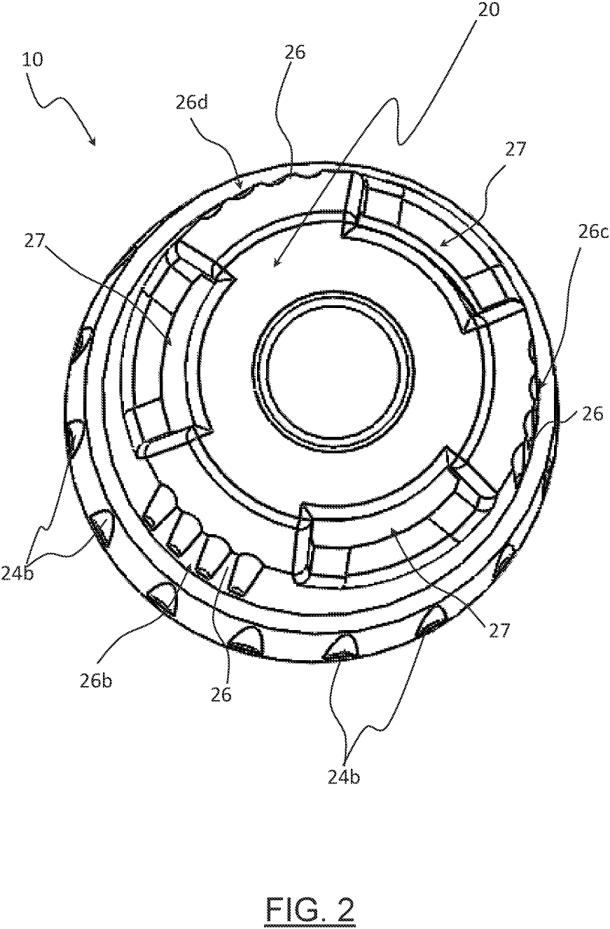

As shown in the non-limiting example of FIG. 2, the second gripping surface elements 26 are grouped in three areas 26b-26d circumferentially equally spaced apart from each other, to form three groups of second gripping surface elements 26.

In particular, the three areas 26b-26d are circumferentially offset from each other by about 120°.

In a variant not shown, the second gripping surface elements 26 are grouped in two areas circumferentially spaced (preferably equally spaced) apart from each other.

As shown in FIG. 2, the main body 20 comprises, at the second cylindrical portion 26a, three through openings 27, each one arranged between two respective groups of second gripping surface elements 26.

In the aforementioned variant not shown, the through openings 27 are two.

Each through opening 27 has an axial extension lower than or equal to 25% of the axial extension of the main body 20.

The through openings 27 are adjacent to the proximal end portion 22 of the main body 20.

As shown in FIG. 2, the through openings 27 have a circumferential extension substantially equal to that of the groups of second gripping surface elements 26. In particular, each through opening 27 and each group of second gripping surface elements 26 has an angular extension equal to about 60°.

As shown in FIG. 4, the radially outer surface 30a of the secondary body 30 comprises a plurality of surface ribs 34, which are four in the non-limiting example shown herein.

The surface ribs 34 are arranged at a proximal cylindrical portion 34a of the main body 20, have an elongated shape, extend substantially parallel to the main axis X and are circumferentially equally spaced apart from each other.

When the main body 20 is coupled to the secondary body 30, the surface ribs 34 contact a radially inner surface 20b of the main body 20, or are in a close proximal position with respect to said radially inner surface 20b. In both cases, a gap is formed between the main body 20, the secondary body 30 and each pair of circumferentially adjacent surface ribs 34.

As shown in FIG. 4, the secondary body 30 comprises a plurality of surface recesses 36, which are four in the non-limiting example shown herein.

Each surface recess 36 is arranged between two respective surface ribs 34.

The surface recesses 36 are provided at a proximal portion of the secondary body 30 that is adjacent to the proximal end portion 32 of the secondary body 30. In particular, the surface recesses 36 extend up to the proximal end portion 32.

The protective cap 10 described above is inserted on the needle of the syringe, which is intended to undergo at least one sterilisation process by ethylene oxide fed with pressure. The latter enters the protective cap 10 through the through openings 27 of the main body 20, is substantially evenly distributed in the gaps formed between the main body 20 and the secondary body 30 and passes through the secondary body 30 made of gas permeable material.

In use, the syringe user removes the protective cap 10 from the syringe needle by moving it in an axial direction after gripping it by placing the fingers on the first gripping surface elements 24 and/or the second gripping surface elements 26. After use, the user may reposition the protective cap 10 on the syringe needle by moving it in an axial direction by placing the fingers on the first gripping surface elements 24 and/or the second gripping surface elements 26.

Of course, those skilled in the art, in order to satisfy specific and contingent requirements, can bring numerous modifications and variants to the present invention as described above, all of them being in any case within the scope of protection defined by the following claims.

1. A protective cap for a syringe needle, comprising a main body extending circumferentially about a main axis and having a predetermined axial extension, wherein the main body comprises first gripping surface elements arranged at a first cylindrical portion of the main body and distributed throughout said first cylindrical portion, circumferentially spaced apart from each other.

2. The protective cap according to claim 1, wherein said first cylindrical portion is a distal portion of the main body with respect to a patient when the protective cap is coupled to the syringe needle and the tip of the syringe needle is oriented towards the patient.

3. The protective cap according to claim 1, wherein each of said first gripping surface elements is arranged between two respective recesses which extend substantially parallel to the main axis.

4. The protective cap according to The protective cap according to claim 1, wherein said first gripping surface elements are circumferentially equally spaced apart from each other.

5. The protective cap according to claim 1, wherein the main body comprises second gripping surface elements arranged at a second cylindrical portion of the main body.

6. The protective cap according to claim 5, wherein said second gripping surface elements extend substantially parallel to the main axis.

7. The protective cap according to claim 5, wherein said second gripping surface elements are grouped in two or three areas circumferentially spaced apart from each other.

8. The protective cap according to claim 7, wherein said areas are circumferentially equally spaced apart from each other.

9. The protective cap according to claim 5, wherein said second cylindrical portion is a proximal portion of the main body with respect to a patient when the protective cap is coupled to the syringe needle and the tip of the syringe needle is oriented towards the patient.

10. The protective cap according to claim 9, wherein the main body comprises, at the second cylindrical portion, two or three through openings, each one arranged between two respective second gripping surface elements.

11. The protective cap according to claim 10, wherein each through opening has an axial extension lower than or equal to 25% of the axial extension of the main body.

12. The protective cap according to claim 1, comprising a secondary body extending circumferentially about said main axis in a radially inner position with respect to said main body and having an axial extension lower than that of said main body.

13. The protective cap according to claim 12, wherein said secondary body comprises a plurality of surface ribs in contact with a radially inner surface of the main body.

14. The protective cap according to claim 13, wherein the ribs of said plurality of surface ribs are substantially parallel to said main axis.

15. The protective cap according to claim 13, wherein the ribs of said plurality of surface ribs are circumferentially equally spaced apart from each other.

16. The protective cap according to claim 15, wherein the secondary body comprises a plurality of surface recesses, each one arranged between two respective ribs of said plurality of surface ribs.
